(19) United States
(12) Patent Application Publication
(10) Pub. No.: US 2022/0023051 A1
(43) Pub. Date: Jan. 27, 2022

(54) Interbody Microstructure Device

(71) Applicants: Nicholas Cordaro, Encinitas, CA (US); Andres Talamantes, Vista, CA (US)

(72) Inventors: Nicholas Cordaro, Encinitas, CA (US); Andres Talamantes, Vista, CA (US)

(21) Appl. No.: 17/381,577

(22) Filed: Jul. 21, 2021

Related U.S. Application Data
(60) Provisional application No. 63/054,933, filed on Jul. 22, 2020.

(51) Int. Cl. A61F 2/34; A61F 2/30

(52) U.S. Cl. CPC A61F 2/34; A61F 2/30767; A61F 2002/30985; A61F 2002/3093; A61F 2/3094

(57) ABSTRACT

A medical device such as an interbody cage component, a hip stem component, or an acetabular shell component having a microstructure in at least one direction for osteointegration and boney in-growth. The microstructure is controlled by machine parameters and may be created by an additive manufacturing program. Typically, the microstructure occurs in both the +/−X and the +/−Y directions, and the microstructure can be added in the range of 0.010 to 0.150 mm.

### CROSS-REFERENCE TO RELATED APPLICATION

The present application claims priority to and the benefit of U.S. Provisional Application No. 63/054,933 filed on Jul. 22, 2020, which is incorporated herein by reference in its entirety.

### FIELD OF THE INVENTION

The present invention relates generally to a medical device such as an interbody cage, hip stem or acetabular shell with at least one area of increased surface topography or texturing. More specifically, the increased surface topography or raised areas of the medical device provide for osteoconduction and bony in-growth while in vivo, thereby enabling faster healing and potentially reducing extended or protracted physical therapy sessions. Accordingly, the present specification makes specific reference thereto. However, it is to be appreciated that aspects of the present invention are also equally amenable to other like applications, devices and methods of manufacture.

### BACKGROUND OF THE INVENTION

By way of background, several surgical techniques have been developed to address various issues with a patient's joints and/or spine, such as disc degeneration and deformity. Thus, spinal fusion has become a recognized surgical procedure for mitigating back pain by restoring biomechanical and anatomical integrity to the spine. Spinal fusion techniques often involve the removal, or partial removal, of at least one intervertebral disc and preparation of the disc space for receiving an implant by shaping or contouring the exposed vertebral endplates to make the endplates more receptive to the placement of the implant. An implant is then inserted between the opposing endplates and the implant is then secured to the end plates to complete the procedure.

Several interbody implant systems have been introduced to facilitate interbody fusion. Traditional implants generally involve a geometrically-shaped implant that is typically packed with bone graft material, and surgically placed within the intervertebral disc space. However, the interbody implant system often shows a lack of implant incorporation with the vertebral bone, as the bone does not fuse with the implant. In most cases, the typical fusion implant is not able to incorporate or integrate with the vertebral bone, even years after implantation, as shifting of the plate makes it difficult for the slow bone regeneration to attach to the plate. The inability for the implant to fuse with the bone persists, despite the use of a variety of different materials to construct the implants. There is a perception that the use of cadaver bone is resorbable and will be replaced by new bone once it resorbs. In contrast, the use of polyetheretherketone (“PEEK”) implants has been reported to become surrounded by fibrous tissue which precludes it from incorporating with surrounding bone. There have also been reports relating to the development of new bio-active materials which can more readily incorporate into bone. The application of such bio-active materials has been limited, however, for several reasons including biocompatibility, structural strength and lack of regulatory approval.

Consequently, there is a long felt need in the art for a medical device that includes the ability for allowing bony in-growth and osteoconduction to allow for the implant incorporation or integration with the vertebral bone. There is also a long felt need in the art for a medical device that is relatively inexpensive to manufacture and that is both safe and easy to use.

More specifically, the present invention discloses a medical implant device such as an interbody cage, hip stem or acetabular shell with distinct areas of increased surface topography to allow for bony in-growth and osteoconduction. Specifically, the interbody cage, hip stem, or acetabular shell includes a microstructure in at least one direction and on at least one area on the device. The microstructure is controlled by equipment (machine) parameters or with the additive manufacturing program in order to build up the surface topography or other raised areas.

While this specification makes specific reference to an interbody cage, hip stem, implant, or acetabular shell of the present invention as comprising microstructures to provide for bony in-growth and osteoconduction, it will be appreciated by those of ordinary skill in the art that aspects of the present invention are also equally amenable to other like applications and/or other such medical devices.

### SUMMARY OF THE INVENTION

The following presents a simplified summary in order to provide a basic understanding of some aspects of the disclosed innovation. This summary is not an extensive overview, and it is not intended to identify key or critical elements or to delineate the scope thereof. Its sole purpose is to present some concepts in a simplified form as a prelude to the more detailed description that is presented later.

The subject matter disclosed and claimed herein, in one aspect thereof, provides for a medical device such as an interbody cage component, a hip stem component, an acetabular shell component or other medical or surgical implant which includes a microstructure in at least one direction, and at least one area of one surface of the implant for osteointegration and bony in-growth. The microstructure is provided in a predetermined pattern having first and second zones of structures. The first zone has more structures than the second zone. The first and second zones can be created by alternating the energy or duration of the additive manufacturing or coating processes in order to create distinct regions. The interbody cage component has a proximal end, a distal end, an interior surface and an exterior surface. Further, the interior surface of the interbody cage component has a pattern of microstructures provided in at least one direction and in at least one area of the interior surface. The microstructure is controlled by machine parameters or the additive manufacturing program to provide areas with more or less microstructures.

In an alternative embodiment, the present invention may comprise an interbody cage component that has a microstructure in both the +/−X and the +/−Y directions, and the surface topography or areas of distinct texture can be added in the range between 0.010 to 0.150 mm.

In another embodiment of the present invention, the microstructure can be controlled by machine parameters. The parameters are set to extend alternating hatch lines and/or to add a secondary alternating contour line out past the original or initial contour line, to give the surface additional controllable macro texture. By creating a distinct pattern of structures or regions on one or more surfaces of the implant, the manufacturing process can be adapted to different types of surgical procedures so as to be able to optimize a configuration that may speed the on-growth of bone material.

In another embodiment, the machine parameters are set to delay when the laser turns off, or initiate the start by one or more microseconds. This keeps the laser in an operating mode while the focus of the laser and the energy moves from one hatch to the next, to create curved or raised extensions. The two pathways can also work in concert with each other, thereby allowing other complex geometries and configurations. The arrangements can be configured depending on the contouring of the vertebral endplates so that the implant is specifically generated for the particular patient.

In a yet further embodiment of the present invention, a method of producing a microstructure device is provided and includes the steps of initially contouring a bone area in a patient and then providing a plate or other implant device for using in a surgical procedure. Next, a design is created for a pattern on the implant or plate based on the step of contouring of the bone area and then forming the pattern on the implant or plate by a variable manufacturing process. The formed pattern is compared with the design and the pattern may be modified based on the step of comparing the formed pattern with the design. Finally, the implant or plate is secured to the contoured bone of a patient.

To the accomplishment of the foregoing and related ends, certain illustrative aspects of the disclosed innovation are described herein in connection with the following description and the annexed drawings. These aspects are indicative, however, of but a few of the various ways in which the principles disclosed herein can be employed and is intended to include all such aspects and their equivalents. Other advantages and novel features will become apparent from the following detailed description when considered in conjunction with the drawings.

### BRIEF DESCRIPTION OF THE DRAWINGS

The description refers to provided drawings in which similar reference characters refer to similar parts throughout the different views, and in which:

FIG. 1A illustrates a perspective view of the macro topography of one potential embodiment of an interbody cage component of the present invention in accordance with the disclosed structure;

FIG. 1B is a cross sectional view of the plate of the present invention which shows the interior surface, exterior surface and the pattern that is built up on the interior surface of the plate;

FIG. 2 shows a perspective view of the macro topography with added texture of one potential embodiment of an interbody cage component of the present invention in accordance with the disclosed specification;

FIG. 3 displays a perspective view of one potential embodiment of the added texture of the present invention shown in a close-up view in accordance with the disclosed description;

FIG. 4 presents a perspective view of one potential alternative embodiment of the added texture to the interbody cage component of the present invention in accordance with the disclosed structure;

FIG. 5 illustrates a perspective view of one potential embodiment of the hatch lines and contour line created during the additive manufacturing program of the present invention in accordance with the disclosed specification;

FIG. 6 shows a perspective view of one potential embodiment of the laser pathways created during the additive manufacturing program of the present invention in accordance with the disclosed description; and

FIG. 7 provides a block diagram showing an exemplary method of making an implant or plate having a microstructure.

### DETAILED DESCRIPTION OF THE INVENTION

The innovation is now described with reference to the drawings, wherein like reference numerals are used to refer to like elements throughout. In the following description, for purposes of explanation, numerous specific details are set forth in order to provide a thorough understanding thereof. It may be evident, however, that the innovation can be practiced without these specific details. In other instances, well-known structures and devices are shown in block diagram form in order to facilitate a description thereof.

A medical or surgical device such as an interbody cage component, hip stem component, implant or an acetabular shell component is presented and comprises a microstructure in at least one direction and on at least one surface for osteointegration and boney in-growth. The microstructure is controlled by machine parameters or the additive manufacturing program and may be varied as needed to fit a particular patient's requirement or bone surface as developed as part of the surgical procedure. Typically, the microstructure occurs in both the +/−X and the +/−Y directions, and the microstructure can be added in the range of a height or thickness of about 0.010 to 0.150 mm. The microstructure is provided in a predetermined pattern having first and second zones of structures. The first zone has more structures than the second zone. The first and second zones can be created by alternating the energy or duration of the additive manufacturing or coating processes in order to create distinct regions. By creating a distinct pattern of structures or regions on one or more surfaces of the implant, the manufacturing process can be adapted to different types of surgical procedures so as to be able to optimize a configuration that may speed the on growth of bone material.

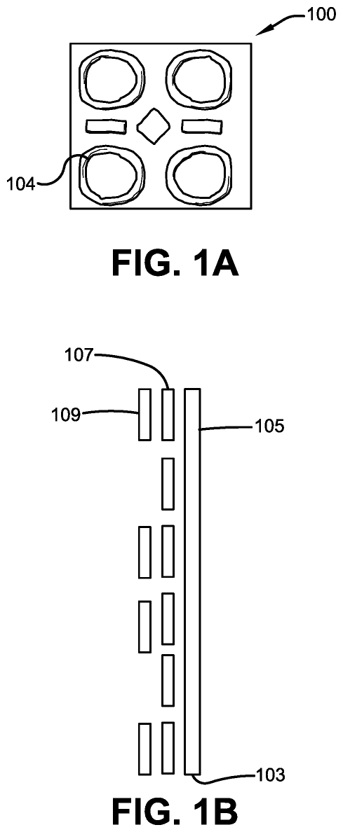

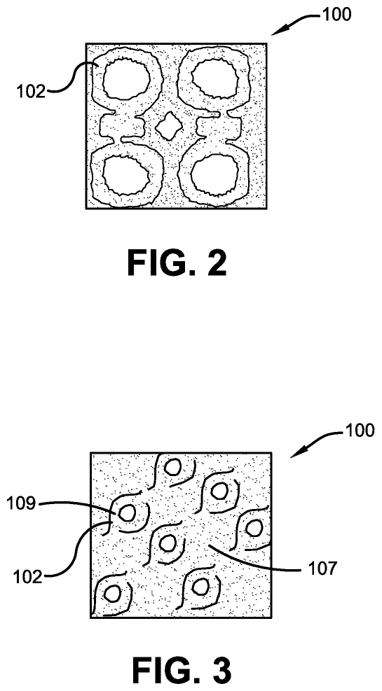

Referring initially to the drawings, FIGS. 1A, 1B and 2 illustrate an interbody cage component, implant or base component 100 comprising a microstructure 102 in at least one direction for osteointegration and boney in-growth. The interbody cage component 100 is typically manufactured using additive manufacturing (AM) techniques and grown as one part on the surface of the plate or implant and is completely integrated with the plate surface so that it does not become dislodged or break apart.

Additionally, the interbody cage component, implant or base component 100 and its components can be any suitable size, shape, and configuration as is known in the art without affecting the overall concept of the invention. One of ordinary skill in the art will appreciate that the shape and size of the interbody cage component 100 as shown in the FIG. 1A is for illustrative purposes only and many other shapes and sizes of the interbody cage component 100 are well within the scope of the present disclosure. Although dimensions of the interbody cage component 100 (i.e., length, width, and height) are important design parameters for good performance, the interbody cage component 100 may be any shape, size or configuration that ensures optimal performance during use.

As shown in FIG. 1A, the interbody cage component, implement or base component 100 comprises a proximal end, a distal end, an interior surface and an exterior surface. Further, the surface of the interbody cage component 100 would include a microstructure 102 in at least one direction and in at least one area. The microstructure 102 is controlled by machine parameters or the additive manufacturing program. Thus, FIG. 1A discloses the surface macro topography 104 of the interbody cage component 100. Typically, macro topography 104 can be controlled with unique lattice structures and CAD designs. The microstructure 102 is then controlled by our equipment (machine) parameters. FIG. 1B shows a cross sectional view of the implant or plate of the present invention showing the interior 103 and exterior 105 of the plate 100. On the interior surface 103, a first layer 107 is applied on the surface and through additive manufacturing a further layer 109 is built up to create different regions or zones on the interior surface of the plate.

As shown in FIG. 2, the interbody cage component, implant or base component 100 comprises a microstructure 102 in both the +/−X and the +/−Y directions. The texture can be added in the range of about 0.010 to 0.150 mm and the height and thickness of the texturing of the surface topography can be varied depending on the requirements of the surgical procedure and or contouring that was done to the bone prior to placement of the plate or implant.

FIG. 3 discloses an enlarged or expanded view of the microstructure 102 in both the +/−X and the +/−Y directions of the interbody cage component 100. More specifically, FIG. 3 depicts both shallow areas 107 or areas where less build up has occurred, and raised areas 109 where more build up has occurred and is raised a further elevation over the height of the base of the plate or cage component 100.

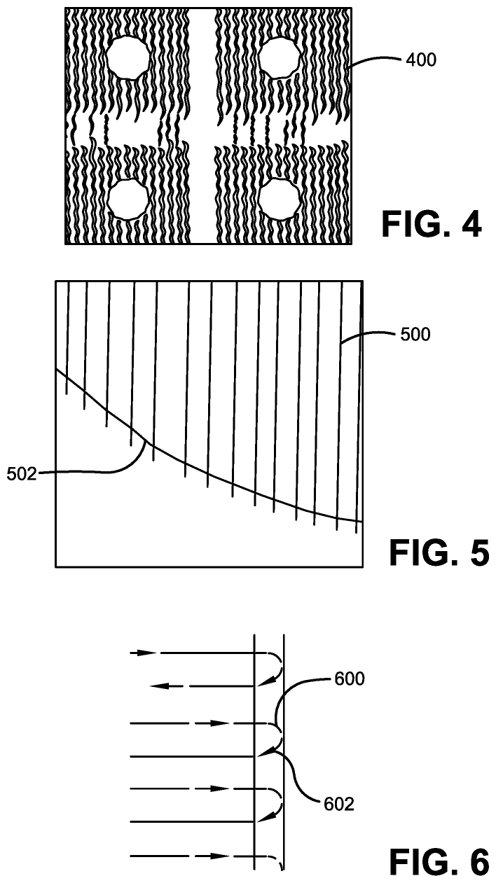

In another embodiment shown in FIG. 4, the microstructure 400 of the present invention is controlled by machine parameters and provides the ability to vary the amount, height, width and other features of the coating or structures to be formed. The parameters are set to extend alternating hatch lines and/or to add a secondary alternating contour line, out past the original contour line, to give the surface additional controllable macro texture and other raised features. One way this is done is disclosed in FIG. 5. More specifically, the hatch lines 500 extend past the contour line 502, as shown in FIG. 5, giving the surface the micro texture 400 shown in FIG. 4.

In another embodiment shown in FIG. 6, the machine parameters are set to delay when the laser turns off or has the laser start early, by microseconds. This keeps the laser on while the focus of the laser moves from one hatch line to the next, creating curved extensions similar to those found with hook and loop fasteners, such as those marketed under the brand Velcro®. Thus, the delay of turning the laser off develops an area 600 and starting the laser early develops an area 602, as shown in FIG. 6. The two pathways can also work in concert with each other while allowing other alternatives and other complex geometries to be created in order to accommodate different bone configurations or surgical procedures. For example, turning the laser on or off can create a J-Hook shape extending out past the surface of the structure, or any other suitable shape as is known in the art can be created by adjusting the laser as well. In another embodiment of the present invention, the medical device can be a hip stem component or acetabular shell component that has a micro surface in both the +/−X and the +/−Y directions. The texture can be added in the range of about 0.010 to 0.150 mm.

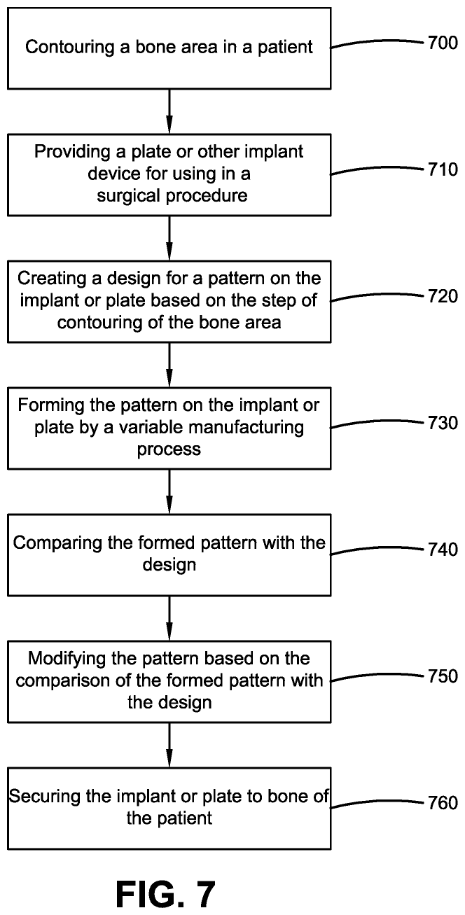

FIG. 7 shows an exemplary block diagram which provides steps in connection with forming a surface topography. The method includes the steps of initially contouring a bone area at step 700 in a patient, then providing a plate or other implant device for using in a surgical procedure at step 710. Next, at step 720 a design for a pattern is created on the implant or plate, based on the step of contouring of the bone area. At step 730, the pattern is formed on the implant or plate by a variable manufacturing process. The formed pattern is compared with the design at step 740 and the pattern may be modified at step 750 based on the step of comparing the formed pattern with the design. Finally, the implant or plate is secured to the contoured bone of a patient at step 760.

What has been described above includes examples of the claimed subject matter. It is, of course, not possible to describe every conceivable combination of components or methodologies for purposes of describing the claimed subject matter, but one of ordinary skill in the art may recognize that many further combinations and permutations of the claimed subject matter are possible. Accordingly, the claimed subject matter is intended to embrace all such alterations, modifications and variations that fall within the spirit and scope of the appended claims. Furthermore, to the extent that the term “includes” is used in either the detailed description or the claims, such term is intended to be inclusive in a manner similar to the term “comprising” as “comprising” is interpreted when employed as a transitional word in a claim.

What is claimed is:

1. A surgical implant comprising:
a base having an interior surface, an exterior surface, a proximal end and a distal end; and
a plurality of microstructures formed on a select one of the interior and exterior surfaces, wherein the plurality of microstructures is formed in at least one direction and in at least one area of the select one of the interior and exterior surfaces.

2. The surgical implant of the present invention as recited in claim 1, wherein the surgical implant is selected from a group consisting of an interbody cage component, a hip stem component, an implant or an acetabular shell component.

3. The surgical implant of the present invention as recited in claim 1, wherein the plurality of microstructures is formed by an additive manufacturing process.

4. The surgical implant of the present invention as recited in claim 1, wherein a portion of the plurality of microstructures are formed on the interior surface of the base.

5. The surgical implant of the present invention as recited in claim 4, wherein the portion is formed in a plurality of zones on the interior surface.

6. The surgical implant of the present invention as recited in claim 5, wherein a first zone of the plurality of zones has a first microstructure having at least one different characteristic from a second microstructure in a second zone of the plurality of zones.

7. The surgical implant of the present invention as recited in claim 6, wherein the at least one different characteristics is a height or a width of the microstructure.

8. The surgical implant of the present invention as recited in claim 1, wherein the at least one direction is selected from a +/−X or a +/−Y direction.

9. The surgical implant of the present invention as recited in claim 8, wherein the plurality of microstructures have a height in a range of about 0.010 to 0.150 mm.

10. The surgical implant of the present invention as recited in claim 1, wherein the plurality of microstructures form a surface topography having a plurality of shallow areas and a plurality of raised areas to create a plurality of distinct zones.

11. A method of producing a microstructure device comprising the steps of:
contouring a bone area in a patient;
providing an implant device for use in a surgical procedure;
creating a design for a pattern of microstructures on the implant device based on the step of contouring of the bone area; and
forming the pattern on the implant device by a variable manufacturing process.

12. The method as recited in claim 11 further comprising a step of comparing the design and the pattern after the step of forming.

13. The method as recited in claim 12 further comprising a step of modifying the pattern based on the step of comparing the formed pattern with the design after the step of forming.

14. The method as recited in claim 13 further comprising a step of securing the implant device to the contoured bone area of the patient after the step of forming.

15. The method as recited in claim 11, wherein the pattern comprises a plurality of microstructures having a height in a range of about 0.010 to 0.150 mm.

16. The method as recited in claim 11, wherein the microstructure device is selected from a group consisting of an interbody cage component, a hip stem component, an implant and an acetabular shell component.

17. The method as recited in claim 11, wherein the step of forming is performed in at least one direction, and further wherein the at least one direction is selected from a +/−X or a +/−Y direction.

18. The method as recited in claim 11, wherein the pattern of microstructures has at least one zone with a different characteristic from at least one other zone, and further wherein the pattern of microstructures is provided on an interior surface of the microstructure device.

19. The method as recited in claim 11, wherein the step of forming the pattern of microstructures creates a surface topography having a plurality of distinct zones comprised of a plurality of shallow and raised areas.

20. An interbody cage component comprising:
a base having an interior surface and an exterior surface;
the interior surface having a surface topography having shallow areas and raised areas and surface topography having microstructures having a height or thickness in the range of about 0.010 to 0.150 mm; and.
the surface topography formed in at least one direction and the at least one direction is selected from +/−X or +/−Y direction on the interior surface.
